(19) United States
(12) Patent Application Publication
(10) Pub. No.: US 2011/0237972 A1
(43) Pub. Date: Sep. 29, 2011

(54) NONINVASIVE MEASUREMENT OF UTERINE EMG PROPAGATION AND POWER SPECTRUM FREQUENCY TO PREDICT TRUE PRETERM LABOR AND DELIVERY

(76) Inventors: Robert Garfield, Goodyear, AZ (US); Rainer J. Fink, College Station, TX (US); Jack N. McCrary, Houston, TX (US)

(21) Appl. No.: 13/069,738

(22) Filed: Mar. 23, 2011

Related U.S. Application Data
(60) Provisional application No. 61/316,460, filed on Mar. 23, 2010.

(51) Int. Cl. A61B 5/0488

(52) U.S. Cl. 600/546

(57) ABSTRACT

A method operable to more accurately predict true preterm labor and delivery is provided. Trans-abdominal uterine electromyography (EMG) and power spectrum (PS) analysis can identify electrical signals characteristic of labor at term and preterm with relatively high positive and negative predictive values. The use of propagation velocity (PV) of uterine EMG signals may either be done independently or in conjunction with PS analysis. This method involves applying at least two pairs of electrodes to a maternal abdomen. The time associated with measuring a voltage spike of a propagating myometrial wave traveling through the pairs of electrodes allows the amount of time required for the propagating myometrial wave to transverse the distance between electrodes to be determined. With this information a propagation velocity (PV) of the propagating myometrial wave may be determined. This PV may be compared to a labor positive predictive value (PPV). A favorable comparison indicates an increased probability of true preterm labor and delivery.

### CROSS REFERENCE TO RELATED APPLICATIONS

The present U.S. Utility Patent Application claims priority pursuant to 35 U.S.C. §119(e) to the following U.S. Provisional Patent Application which is hereby incorporated herein by reference in its entirety and made part of the present U.S. Utility Patent Application for all purposes:

a. U.S. Provisional Application Ser. No. 61/316,460, entitled “NONINVASIVE MEASUREMENT OF UTERINE EMG PROPAGATION AND POWER SPECTRUM FREQUENCY TO PREDICT TRUE PRETERM LABOR AND DELIVERY,” filed Mar. 23, 2010.

### TECHNICAL FIELD OF THE INVENTION

Embodiments of the present invention relate generally to detection of Uterine EMG Propagation, and, more particularly, embodiments of the present invention relate to a means of Predicting True Preterm Labor and Delivery.

### BACKGROUND OF THE INVENTION

Spontaneous preterm labor and consequent preterm birth remains as the biggest unsolved obstetrical problem. Approximately 5000 infants die each year in the United States from complications of prematurity and infants born preterm who survive are more likely to develop visual and hearing impairment, chronic lung disease, cerebral palsy, and delayed development in childhood. Although the perinatal mortality rate due to prematurity has decreased dramatically over the past four decades in high-income countries, this reduction has resulted from improvements in neonatal care for premature babies and has occurred in spite of the increasing incidence of premature delivery. Most women who deliver preterm have no apparent risk factors, every pregnancy should therefore be considered to be potentially at risk.

Once preterm labor is established, none of the currently available treatments and interventions can prolong pregnancy sufficiently to allow further intrauterine growth and maturation. The key to treating preterm labor today is its early detection and institution of treatment before any benefit from tocolytic therapy is already lost. This permits delaying delivery long enough to transfer the pregnant patient to the most appropriate hospital, administer corticosteroids and prophylactic antibiotics to reduce neonatal morbidity and mortality.

Accurate early diagnosis of preterm labor is, however, a major problem. Today, up to 50% of patients diagnosed with preterm labor are not actually in preterm labor and as many as 20% of symptomatic patients diagnosed as not being in labor will deliver prematurely. The diagnosis of preterm labor still often relies on presence of contractions. However, contractions occur commonly in normal pregnancy and their detection through maternal self perception and/or tocodynamometry (TOCO) has a low sensitivity and positive predictive value for preterm delivery. Cervical dilation, effacement, consistency, position, and station of the presenting part, determined by manual examination are components of the Bishop scoring system which is also used clinically as a predictor of preterm delivery. But the assessement of the cervix by digital exam is subjective and its prognostic values have also been shown to be low. There is now substantial evidence that measuring the cervical length by transvaginal ultrasound and testing for fetal fibronectin in cervicovaginal fluid can help to avoid unnecessary treatment due to the high negative predictive values of these tests. Their positive predictive values are, however, low and many patients with short cervix and positive fibronectin do not deliver preterm.

During the latter stages of pregnancy and during the actual laboring process, two methods of acquiring and monitoring uterine activity are generally used. The first method involves the use of a tocodynamometer. Toco is a non-invasive device fastened to the external abdomen of the patient that is used to measure uterine contraction frequency. The typical toco consists of an external strain-gauge or pressure transducer designed to measure the stretch of the patient's stomach to determine when a uterine contraction has occurred. When the skin stretches, the pressure transducer records an electrical signal whose waveform can be evaluated and correlated to the stage or phase of labor by the treating physician.

The toco, however, has many drawbacks. One disadvantage is that it is an indirect method of pressure reading and is therefore subject to many interfering influences which can falsify the measuring result. Its effectiveness can be entirely dependent on the tightness of the belt used to place the toco on the maternal abdomen. Also, the effectiveness of the toco is dependent on transducer location and, therefore, does not function once the baby has descended down the uterus and into the birth canal where no pressure transducer is present to report pressure variations. Moreover, the toco is highly inaccurate and fails to function properly on heavier patients since the pressure transducer requires that uterine contractions be transmitted through whatever intervening tissues there may be to the surface of the abdomen.

The second method of acquiring and monitoring uterine activity involves the use of an intrauterine pressure catheter (“IUPC”). A typical IUPC consists of a thin, flexible tube with a small, tip-end pressure transducer that is physically inserted into the uterus next to the baby. The IUPC is configured to measure the actual pressure within the uterus and thereby indicate the frequency and intensity of uterine contractions. However, in order to place the IUPC, the amniotic membrane must be ruptured so that the catheter can be inserted. Improper placement of the IUPC catheter can result in false readings. Similarly, the catheter opening can become plugged and provide false information requiring the removal, cleaning and reinsertion of the IUPC, Lastly, inserting the catheter runs the risk of injuring the head of the baby, and also carries with it a significant infection risk. Thus, generally the IUPC is rarely used, and can only be used at delivery.

### SUMMARY OF THE INVENTION

Embodiments of the present disclosure are directed to systems and methods that are further described in the following description and claims. Advantages and features of embodiments of the present disclosure may become apparent from the description, accompanying drawings and claims.

Embodiments of the present disclosure provide a system or methodology that overcomes the above-noted disadvantages of the toco and IUPC. In particular, embodiments of the present disclosure provide a system that both overcome the inaccuracy of the toco and the invasive and precarious nature of the IUPC.

Embodiment of the present disclosure provides a method operable to more accurately predict true preterm labor and delivery. This method involves applying at least one pair of electrodes to a maternal abdomen. The time associated with measuring a voltage spike of a propagating myometrial wave traveling through the pair of electrodes allows the amount of time required for the propagating myometrial wave to transverse the distance between electrodes to be determined. With this information a propagation velocity (PV) of the propagating myometrial wave may be determined. This PV may be compared to a labor positive predictive value (PPV). A favorable comparison indicates an increased probability of true preterm labor and delivery. The propagating myometrial wave may be detected using electrodes to detect a uterine electromyography (EMG) signal associated with the propagating myometrial wave. This increased probability of true preterm labor may especially indicate and favorably predict delivery within seven days. In addition to the PV signal a power spectrum signal may be measured and used to determine the increased probability of true preterm labor and delivery. The power spectrum signal may be analyzed for peak and median frequency, peak and medium amplitude, restoration, inter burst interval duration, and standard deviation of interbursed interval duration. Additionally embodiments of the present disclosure may allow for the correction of the detected propagating myometrial wave using skin impedance matching.

Yet another embodiment of the present disclosure by the system operable to predict true preterm labor and delivery. The system includes two or more pairs of electrodes associated with a sensing module and a signal processing module. The pairs of electrodes may be placed in communication with a maternal abdomen. The pairs of electrodes may be used to acquire a multitude of raw uterine electromyography signals associated with the propagating myometrial wave in multiple directions with respect to the orientation of the uterus. The signal processing module coupled to the sensing module and the pairs of electrodes may be operable to filter and amplify the raw uterine EMG signals in order to produce processed EMG signals. The signal processing module may then calculate a propagating velocity of the propagating myometrial wave through pair wise comparisons and then compare the PV or the propagating myometrial wave to a labor of positive predictive value wherein a favorable comparison indicates a greatly increased probability of true preterm labor and delivery. The signal processing module would then be able to display to a user or by another means communicate to a user the increased probability of true preterm labor and delivery.

Embodiments of the present disclosure provide a method with a high positive predictive value for preterm delivery that may accurately identify patients in true preterm labor who will benefit from early commencement of tocolytic therapy. Such a method is also extremely valuable in further research of potential treatments for preterm labor. Such research has been largely hindered by the inability to reliably distinguish patients in true preterm labor from patients in false labor who will not deliver preterm regardless of treatment.

Today, there is no accepted method to accurately diagnose true preterm or term labor. Trans-abdominal uterine electromyography (EMG) and power spectrum (PS) analysis can identify electrical signals characteristic of labor at term and preterm with relatively high positive and negative predictive values. The use of propagation velocity (PV) of uterine EMG signals either independently or in conjunction with PS for diagnosing preterm labor has not been reported yet.

Various embodiments of the present disclosure analyze various EMG parameters to predict preterm delivery. Parameters of the power density spectrum may be used to evaluate the effectiveness of uterine contractions, and as such an indicator of labor or progression toward successful delivery. These parameters include peak frequency of the PS, area under the PS curve, individual frequency components of the PS as well as relationships between components of the PS. The inclusion of data obtained from the raw EMG analysis, including PV, EMG burst amplitude, burst duration, and inter-burst duration can be used to further refine the estimate of true versus false labor, resulting in an analysis technique which utilizes two different analysis modalities to obtain a more accurate evaluation of the status of labor. The further combination of the EMG based sensing modality (including all possible analysis mentioned above) with analysis of the cervical status using either new instruments such as the SureTouch® collascope, which measures the ripening of the cervix through Light-Induced Auto Fluorescence, or older technologies such as the Bishops Score, or measurement of the cervical length using ultrasound, results in yet a clearer understanding of the status of labor.

### BRIEF DESCRIPTION OF THE DRAWINGS

For a more complete understanding of the present disclosure and the advantages thereof, reference is now made to the following description taken in conjunction with the accompanying drawings in which like reference numerals indicate like features and wherein:

FIG. 1 illustrates a system for acquiring and processing uterine electromyography (“EMG”) signals in accordance with embodiments of the present disclosure; and

FIG. 2 illustrates an embodiment of the circuit board located in the signal processing module, as described in FIG. 1 in accordance with embodiments of the present disclosure.

FIG. 3 shows that the concept of measuring PV using uterine EMG involves first noting the time difference between associated voltage spikes at two different locations due to a propagating myometrial wave traveling from one location to the other in accordance with embodiments of the present disclosure;

FIG. 4 shows that the measured PV was significantly higher (P<0.001) in labor (31.25±14.91 cm/s) compared with non-labor patients (11.31±2.89 cm/s) in accordance with embodiments of the present disclosure;

FIG. 5A illustrates a comparison of EMG propagation velocity values for preterm patients delivering within 7 days of measurement with those delivering more than 7 days from measurement;

FIG. 5B illustrates a comparison of EMG power spectrum (PS) peak frequency values for preterm patients delivering within 7 days of measurement with those delivering more than 7 days from measurement;

FIG. 6 illustrates EMG propagation velocity increased as the measurement-to-delivery interval decreased in accordance with embodiments of the present disclosure;

FIG. 7 illustrates a comparison of ROC curves for EMG parameters (combination of propagation velocity (PV) and PS peak frequency) and currently used methods to predict preterm delivery within 7 days in accordance with embodiments of the present disclosure;

FIG. 8 illustrates a comparison of skin-electrode impedance measured before EMG recording between false positive (FP, N=0)+false negative (FN, N=6) and true positive (TP, N=14)+true negative (TN, N=68) groups (as determined by measurements of propagation velocity and PS peak frequency)

FIG. 9 illustrates that there is no significant correlation between skin-electrode impedance and patient's BMI; and

FIG. 10 provides a logic flow diagram of a method of predicting true preterm labor and delivery in accordance with embodiments of the present disclosure.

The present disclosure is best understood from the following detailed description when read with the accompanying FIGs., as presented within the text of this application. It is emphasized that, in accordance with the standard practice in the industry, various features are not drawn to scale. In fact, the dimensions of the various features may be arbitrarily increased or reduced for clarity of discussion.

### DETAILED DESCRIPTION OF THE INVENTION

Embodiments of the present disclosure are illustrated in the FIGs., like numerals being used to refer to like and corresponding parts of the various drawings. The following disclosure describes several exemplary embodiments for implementing different features, structures, or functions of the disclosure. Exemplary embodiments of components, arrangements, and configurations are described below to simplify the present disclosure; however, these exemplary embodiments are provided merely as examples and are not intended to limit the scope of the disclosure. Additionally, the present disclosure may repeat reference numerals and/or letters in the various exemplary embodiments and across the FIGs. provided herein. This repetition is for the purpose of simplicity and clarity and does not in itself dictate a relationship between the various exemplary embodiments and/or configurations discussed in the various FIGs. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact. Finally, the exemplary embodiments presented below may be combined in any combination of ways, i.e., any element from one exemplary embodiment may be used in any other exemplary embodiment, without departing from the scope of the disclosure.

Additionally, certain terms are used throughout the following description and claims to refer to particular components. As one skilled in the art will appreciate, various entities may refer to the same component by different names, and as such, the naming convention for the elements described herein is not intended to limit the scope of the disclosure, unless otherwise specifically defined herein. Further, the naming convention used herein is not intended to distinguish between components that differ in name but not function. Further, in the following discussion and in the claims, the terms “including” and “comprising” are used in an open-ended fashion, and thus should be interpreted to mean “including, but not limited to.” All numerical values in this disclosure may be exact or approximate values unless otherwise specifically stated. Accordingly, various embodiments of the disclosure may deviate from the numbers, values, and ranges disclosed herein without departing from the intended scope. Furthermore, as it is used in the claims or specification, the term “or” is intended to encompass both exclusive and inclusive cases, i.e., “A or B” is intended to be synonymous with “at least one of A and B,” unless otherwise expressly specified herein.

In at least one embodiment of the present disclosure, a method for noninvasive measurement of uterine EMG propagation and power spectrum frequency to predict true preterm labor and delivery is provided. One apparatus to be used to obtain the EMG measurements is described in commonly owned U.S. patent application Ser. No. 12/696,936, entitled System and Method or Acquiring and Displaying Uterine EMG Signals, the entire contents of which are hereby incorporated by reference in to this patent application. Another apparatus that may be used to obtain the measurements described herein that utilizes wireless signal transmission methods may be found in commonly owned United States Patent Application entitled System and Method of Acquiring Uterine EMG Signals, Attorney Docket No. RRT-004, the entire contents of which are hereby incorporated by reference in to this patent application.

In proving the validity and accuracy of the method, uterine EMG was recorded from the abdominal surface for 30 minutes at 4 separate recording sites in 116 patients (group 1: term labor, n=22; group 2: term non-labor, n=6; group 3: preterm labor, n=20; group 4: preterm non-labor, n=68). The patients in groups 3 and 4 (n=88) were originally admitted to our institution with the diagnosis of preterm labor at less than 34 weeks gestation. PV was estimated from the time interval between signal arrivals at adjacent electrode pairs. Electrical “bursts” were analyzed by PS from 0.34 to 1.00 Hz. PS peak and median frequency, PS peak and median amplitude, burst duration, interburst interval duration, and standard deviation of burst and interburst interval duration were also analyzed in preterm patients. Student's t-test was used on all of these parameters to compare delivery within (≦), vs. outside of (>), 7 days from the measurement in preterm patients (P<0.05 significant). PV in term patients delivering within, vs. outside of, 24 hours from the measurement was also compared. Predictive values of EMG, Bishop Score, contractions on toco-gram and trans-vaginal cervical length for prediction of preterm delivery were estimated using receiver operator characteristics analysis.

The results of the process of proving method were that the PV was significantly higher (P<0.001) in term labor (31.25±14.91 cm/s) compared with term non-labor patients (11.31±2.89 cm/s). PV and peak PS frequency were significantly higher in preterm patients delivering within 7 days (P<0.05). In ROC analysis to distinguish between preterm labor and non-labor patients, the area under the curve (AUC) value for PV was the highest at 4 days to delivery (AUC=0.69), while for peak PS frequency, the AUC value was the highest at 7 days from delivery (AUC=0.78). A combination (rescaled sum) of PV and PS peak frequency had the best predictive values at 7 days to delivery than for any parameter alone at any time point with a 70% sensitivity, 100% specificity, 100% positive predictive value (PPV) and 90% negative predictive value (NPV). Bishop Score, presence of contractions on tocogram, and cervical length had AUC of 0.72, 0.67, and 0.54, respectively.

Therefore, the changes in the electrical properties of the myometrium including propagation and frequency indicate that significant increases in cellular electrical coupling and excitation occur prior to true preterm labor and also during term labor. PV and PS peak frequency, used jointly, are more predictive of preterm delivery than either variable alone. EMG is more accurate in distinguishing between true and false preterm labor than other methods used clinically.

Myometrial cells are coupled together electrically by gap junctions that provide channels of low electrical resistance between cells and facilitate efficient conduction of action potentials. Throughout most of pregnancy these cell-to-cell channels or contacts are few, indicating poor coupling and decreased electrical conductance. This condition favors quiescence of the myometrium and the maintenance of pregnancy. Before delivery at term or preterm, however, the cell junctions increase and form an electrical syncytium required for effective contractions. Uterine electromyography (EMG) yields valuable information about the changes in the electrical properties of the myometrium. Thus, embodiments of the present disclosure monitor uterine EMG non-invasively from the abdominal surface. Changes in several EMG parameters can indicate the onset of labor. Further, embodiments of the present disclosure use of propagation velocity (PV) of uterine EMG signals for diagnosing preterm labor.

A study was conducted to investigate whether uterine EMG can be used to evaluate PV of uterine electrical signals in labor and non-labor patients at term and preterm. The study then compared predictive values of various EMG parameters, including PV, among one another and also compared to methods currently used in the clinic to predict preterm delivery.

The methodology of the study will not be described. To begin, 116 pregnant women were included in the study at a single institution (St. Joseph's Hospital and Medical Center, Department of Obstetrics and Gynecology, Phoenix, Ariz.): 28 at term (>37 weeks of gestation) and 88 preterm (<34 weeks of gestation). 22 of these women delivered within 24 hours from the EMG measurement (defined as term labor patients) and 6 delivered outside of 24 hours from the measurement (term non-labor).

Preterm patients were admitted with the diagnosis of threatened preterm labor at less than 34 weeks of gestational age. 20 patients delivered within 7 days from the EMG measurement (preterm labor) and 68 did not (preterm non-labor). Calculation of gestational age was based on the last menstrual period and confirmed or modified by ultrasound imaging within the first trimester. All women provided written informed consent for study participation. Data from patients who ultimately underwent cesarean section were not used for analysis. The St. Joseph's Hospital and Medical Center Institutional Review Board approved the study.

Uterine EMG signal recordings were used in the study as described below. Prior to uterine EMG measurement, electrode attachment sites were prepared by cleaning away excess oil with alcohol prep pads in order to improve electrical conduction to the electrode. Ag₂Cl electrodes (Quinton, Bothell, Wash.) were then placed upon the abdominal surface. A standard 4-electrode arrangement was used: symmetric about the navel, with vertical and horizontal axes parallel to the patient vertical and horizontal axes, respectively, and with center-to-center distances between adjacent electrodes set at approximately 5.0 to 5.5 cm apart. The electrodes remained for 10 minutes on the patient, and thereafter electrode-skin impedance measurements were made at the abdominal sites prior to the EMG recording. Impedance measurements were also made immediately following the recording. Uterine EMG was measured for 30 minutes using a custom-built uterine EMG patient-monitoring system manufactured by Reproductive Research Technologies, of Houston, Tex. Patients were asked to remain still while supine without disturbing any of the probes and wires for the recordings. The impedance measurements were obtained using the apparatus and methods described in commonly owned U.S. patent application Ser. No. 12/114,490, entitled Skin Impedance Matching System and Method for Skin/Electrode Interface, the entire contents of which are hereby incorporated by reference in to this patent application.

The received signals were processed and analyzed as described below. Analog EMG signals were digitally filtered to yield a final band-pass of 0.34 to 1.00 Hz, in order to exclude most components of motion, respiration, and maternal and fetal cardiac signals from the analysis, and to more clearly discern “bursts” of uterine electrical activity associated with contractile events. Data were sampled at 100 Hz (this high sampling rate was chosen so as to increase the resolution of power-spectral analysis later). Chart 5 software (ADInstruments, Castle Hill, Australia) was utilized for the signal analysis.

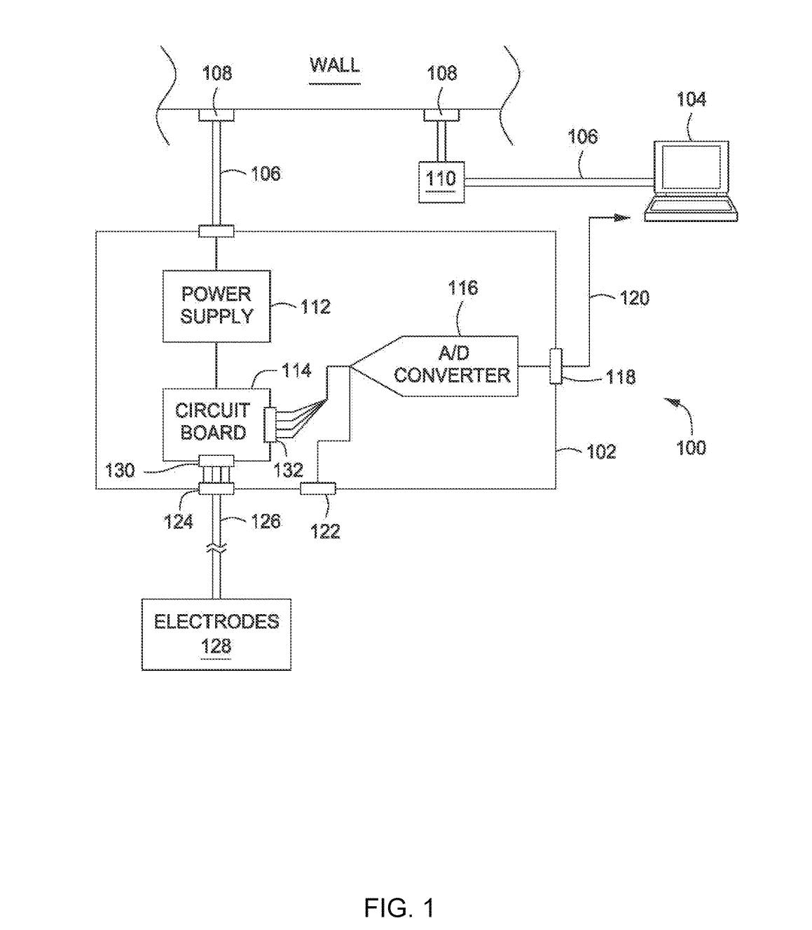

Referring to FIG. 1, illustrated is a system 100 for acquiring and processing uterine electromyography (“EMG”) signals (also sometimes termed electrohystography or EHG). A uterine EMG signal is the functional equivalent to a uterine activity signal created by a toco or IUPC, but can be a great deal more precise. As explanation, uterine contractions comprise coordinated contractions by individual myometrial cells of the uterus. These global muscle contractions are triggered by an action potential and can be seen externally as an EMG signal. When electrodes are placed on the maternal abdomen, they measure the global muscle firing of a uterine contraction, thereby resulting in a “raw” uterine EMG signal.

The system 100 may include a signal processing module 102 communicably coupled to a computer 104. The signal processing module 102 and the computer 104 may each include hardware, however, the computer 104 may include software for executing machine-readable instructions to produce a desired result. In at least one embodiment, the software may include an executable software program created in commercially-available LABV1EW®. The hardware may include at least processor-capable platforms, such as 5 client-machines (also known as personal computers or servers) and hand-held processing devices (such as smart phones, personal digital assistants (PDAs), or personal computing devices (PCDs), for example). Further, hardware may include any physical device that is capable of storing machine-readable instructions, such as memory or other data storage devices. Other forms of hardware include hardware sub-systems, including transfer devices such as modems, modem cards, ports, and port cards. In short, the computer 104 may include any other micro processing device, as is known in the art. The computer 104 may include a monitor for displaying processed uterine EMG signals for evaluation.

In an exemplary embodiment, the computer 104 may include, without limitation, a desktop computer, laptop computer, or a mobile computing device. Moreover, the computer 104 may include a CPU and memory (not shown), and may also include an operating system (“OS”) that controls the operation of the computer 104. The OS may be a MICROSOFT® Windows OS, but in other embodiments, the OS may be any kind of operating system, including without limitation any version of the LINUX® OS, any version of the UNIX® OS, or any other conventional OS as is known in the art.

Both the signal processing module 102 and the computer 104 may be powered via a medical-grade power cord 106 that may be connected to any typical wall outlet 108 conveying 120 volts of power. As can be appreciated, the system 100 may also be configured to operate on varying voltage systems present in foreign countries. For the computer 104, however, the power cord 106 may include an interim, medical-grade power brick 110 configured to reduce or eliminate leakage current originating at the wall outlet 108 that may potentially dissipate through the internal circuitry of the system 100 or a patient.

The signal processing module 102 may house a power supply module 112, a circuit board module 114, and an analog to digital (“A/D”) converter 116. The power supply module 112 may be configured to supply power for the signal processing module 102. In particular, the power supply module 112 may receive 120V-60 Hz power from the wall outlet 108 and convert that into a 12 volt direct current to be supplied to the circuit board module 114. In alternative embodiments, the power supply module 112 may be configured to receive varying types of power, for example, DC current from a battery or power available in foreign countries. As will be described in more detail below, the circuit board 114 may be any type of electronic circuit and configured to receive, amplify, and filter the incoming uterine signals.

The A/D converter 116 may digitize the incoming analog uterine signals into a viewable digital signal transmittable to the computer 104 for display. Specifically, the A/D converter 116 may be communicably coupled to an external USB port 118 located on the body of the signal processing module 102. In an exemplary embodiment, the USB port 118 may connect to a commercially-available USB 6008 (DAQ), available through NATIONAL INSTRUMENTS®. A double-ended USB connection cable 120 may be utilized to communicably couple the USB port 118 to the computer 104. As can be appreciated, however, the disclosure also contemplates alternative embodiments where the USB port 118 may be replaced with a wireless adapter and signal transmitter to wirelessly transmit the processed uterine data directly to a receiver located on the computer 104.

The signal processing module 102 may also include a toco communication port 122 through which physicians may be able to acquire and process uterine signals via a tocodynamometer (“toco”) or IUPC, as is already well-known in the art. For example, through the toco communication port 122, physicians may be able to track maternal and fetal heart rates, and also acquire intrauterine pressures via an IUPC or chronicle uterine activity via a toco. The analog signals sent to the toco communication port 122 may be directed to the A/D converter 116 to be digitized and subsequently displayed through the computer 104.

As described above, the digitized signals may be routed to the computer 104 via the USB port 118 and double-ended USB connection cable 120. Similarly, and more importantly for the purposes of the present disclosure, the signal processing module 102 may include an EMG communication port 124 which may be communicably coupled to at least one pair of electrodes 128 and a patient ground electrode via an EMG channel 126. Through the electrodes 128, physicians may acquire and process raw uterine EMG signals. Specifically, the electrodes 128 may be configured to measure the differential muscle potential across the area between the two electrodes 128 and reference that potential to patient ground. Once the muscle potential is acquired, the raw uterine EMG signal may then be routed to an input 130 for processing within the circuit board 114, as will be described below.

After processing within the circuit board 114, the processed uterine EMG signal may be directed out of the circuit board 114, through an output 132, and to the A/D converter 116 where the analog uterine EMG signal may be subsequently digitized for display on the computer 104. The digitized uterine EMG signal may be transmitted to the computer 104 via the USB port 118 and double-ended USB connection cable 120, as described above. However, alternative embodiments contemplate transmitting the data wirelessly to the computer 104 via a wireless adapter and signal transmitter (not shown). In at least one embodiment, the processed uterine EMG signal may provide uterine contraction frequency and duration information.

Although only one EMG channel 126 is illustrated, the disclosure fully contemplates using multiple EMG channels 126—each EMG channel 126 being communicably coupled to a separate pair of electrodes 128. In an exemplary embodiment, there may be four or more separate EMG channels 126 entering the EMG communication port 124.

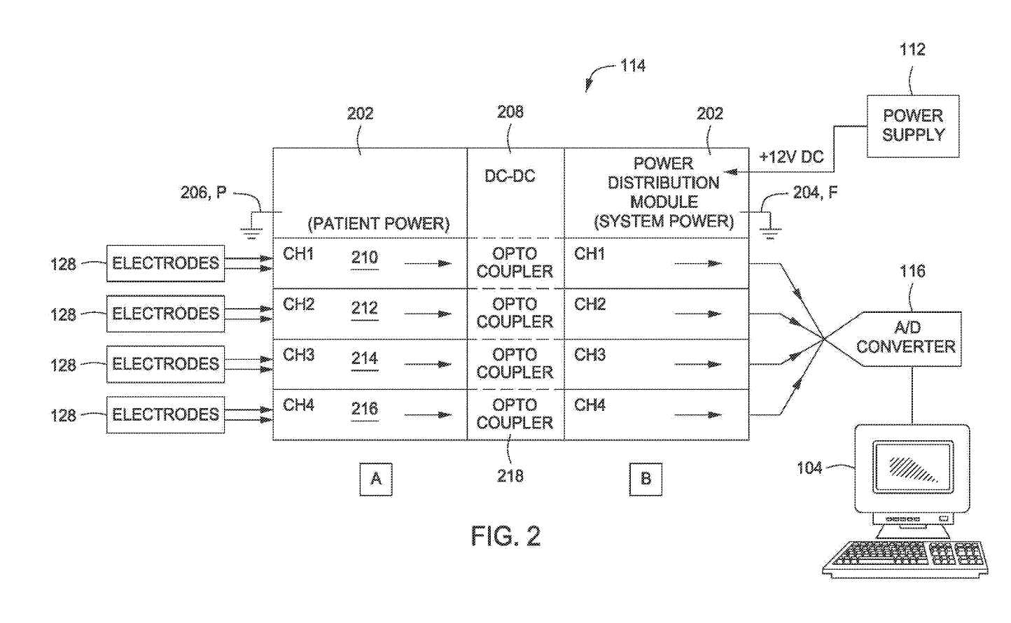

Referring now to FIG. 2, illustrated is an exemplary embodiment of the circuit board 114 located in the signal processing module 102, as described in FIG. 1. The circuit board 114 may include a patient side A, and a wall side B. As explained above, the circuit board 114 may receive a 12V direct current from the power supply module 112. In particular, the power supply module 112 may be communicably coupled to a power distribution module 202 located within the circuit board 114, wherein the power distribution module 202 may be configured to supply varying amounts of voltage to the internal circuitry of the circuit board 114. The power distribution module 202 may include a wall ground 204 and a patient ground 206, designed to not only protect the patient from stray leakage current but also to protect the internal circuitry from overload, as described below.

To help facilitate electrical shock protection for both the patient and the circuitry, the circuit board 114 may include an isolation DC-DC converter 208, or a transformer that separates the patient side A from the wall side B. In exemplary operation, the isolation DC-DC converter 208 may be configured to isolate power signals, thereby preventing stray charges from crossing over from one side and causing damage on the opposite side. In at least one embodiment, the isolation DC-DC converter 208 may include a commercially-available PWR1300 unregulated DC-DC converter.

As illustrated in FIG. 2, the circuit board 114 may be divided into a series of channels 210, 212, 214, 216. In the exemplary illustrated embodiment, four channels 210, 212, 214, 216 are indicated, labeled as CH1, CH2, CH3, and CH4, respectively, and may extend across both patient side A and wall side B. Each channel 210, 212, 214, 216 may be communicably coupled to a pair of electrodes 128, as described above. Once the “raw” uterine EMG signal is obtained by the electrodes 128, the differential signal is then delivered to each respective channel 210, 212, 214, and 216 for processing

Although four separate channels 210, 212, 214, 216 are herein disclosed, alternative embodiments may include more or less than four. In fact, suitable results may be achieved by employing a single-channel configuration. However, since inaccurate EMG signals can often result from poor skin impedance or misplacement of the electrodes 128, a plurality of channels 210, 212, 214, 216 may afford the physician with a plurality of opportunities to acquire an accurate uterine EMG signal. Furthermore, each channel 210, 212, 214, 216 may be separately-viewable on the computer 104 (FIG. 1) after signal processing has taken place.

Similar to the power distribution module 202, as a precautionary measure the channels 210, 212, 214, 216 on patient side A are isolated from their counterpart channels 210, 212, 214, 216 on wall side B by a linear optocoupler 218. In an exemplary embodiment, the linear optocoupler 218 may consist of a commercially-available IL300 optocoupler, available through VISHAY SEMICONDUCTORS®. As can be appreciated to those skilled in the relevant art, the linear optocoupler 218 may serve to avert potential electrical damage to the circuit 114 and the patient (not shown), as leakage current will be prohibited from transferring from one side A,B to the other B,A, or vice versa.

In exemplary operation, the linear optocoupler 218 may be configured to receive a partially processed EMG signal from the patient side A and create an optical light signal that transmits across the linear optocoupler 218 to the wall side B. To be able to optically transmit a signal across the linear optocoupler 218 from the patient side A to the wall side B, the incoming raw uterine EMG signal must first be amplified and filtered, as will be described in detail below. At the wall side B, the optical signal may then be converted back into an electrical signal and then undergo final amplification and filtration processes, as will also be described below. After final amplification and filtration on the wall side B, the processed uterine EMG signal may then be transmitted to the A/D converter 116 where the signal is digitized for display on the computer 104 (FIG. 1).

For better understanding of various embodiments of the disclosure, the following definitions are helpful:

Previously Described EMG Parameters—PS peak frequency, PS median frequency, PS peak amplitude, PS median amplitude, mean burst duration, mean inter-burst interval duration, and standard deviation of burst and inter-burst interval duration were analyzed. PS analysis was performed as described in this group's previous publications.

Propagation Velocity Analysis—The concept of measuring PV using uterine EMG involves first noting the time difference between associated voltage spikes at two different locations due to a propagating myometrial wave traveling from one location to the other. PV can be calculated by dividing the distance (D) that the propagating wave travels by the amount of time (T) required for the propagating wave to traverse this distance.

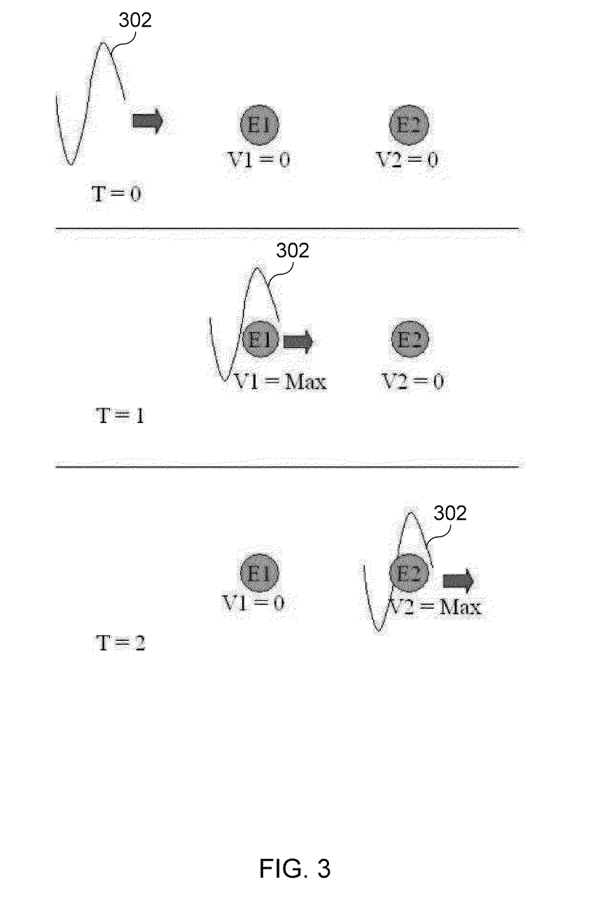

FIG. 3 shows that the concept of measuring PV using uterine EMG involves first noting the time difference between associated voltage spikes at two different locations due to a propagating myometrial wave traveling from one location to the other. For example, assume a propagating myometrial wave 302 originates at time T=0, is thereafter located at electrode pair 1 (E1) at time T=1, and is thereafter located at electrode pair 2 (E2) at T=2. T1-T2 represents the propagation time interval between the signal arrivals at the two locations or travels the distance between the electrode pairs. The methodology for producing an accurate assessment of the average value of [T1-T2] (i.e. TAvg) is to look at all the time differences in corresponding action potential peaks at E1 and E2 for each burst of action potentials, and then to take the average of absolute values of all time differences for bursts in a patient's uterine EMG recording. For the EMG instrument, differential, bipolar electrode pairs were used. Because of this, the propagation may be sensed by finding T2-T1 at adjacent electrode pairs, rather than at individual electrodes. One disadvantage of such a bipolar setup is that purely vertical propagation produces a minimal measurement, due to the common mode rejection of the amplifiers, while purely horizontal waves are registered, and these “horizontally-moving” waves impinge at adjacent upper and lower pairs in rapid succession, thus contributing to an overestimation of the propagation velocity. However, the advantage of a differential bipolar setup over a mono-polar setup is signal quality, allowing us to more accurately identify individual peaks. Only the most prominent bursts were used in these calculations, in order to clearly see and compare peaks at adjacent electrodes. The apparatus and methods used to measure the propagation velocity is shown and described in commonly owned U.S. Provisional Patent Application Ser. No. 61/301,271, entitled Measuring and Displaying the Propagation Velocity of Uterine Action Potentials to Determine the Onset of Labor, the entire contents of which are hereby incorporated by reference in to this patent application.

Skin-electrode impedance—Impedance between the skin and the electrodes and the signal noise as a result of transient electrical potentials between the skin and the electrodes is another critical consideration for processing uterine EMG signals acquired non-invasively from surface mounted electrodes. If the uterine signals are buried within too much noise, then they are much less useful for prognosticating the patient condition. From our own observations, skin-electrode impedance is significantly correlated (negatively) with signal/noise ratio in the uterine EMG traces acquired and tends to fall off as a function of time. Electrode-skin impedance was measured prior to each EMG recording in this study. Impedance measurements were also made immediately following the recording. In order to determine to what extent skin-electrode impedance can affect the value of EMG in predicting preterm delivery the study performed a comparison between impedance values in true negative and true positive vs. impedance values of false negative and false positive patients (as determined by measurements of PV and PS frequency). The study also analyzed the correlation between skin-electrode impedance and patient's BMI. The skin impedance measurements discussed herein were obtained using the apparatus and methods described in commonly owned U.S. patent application Ser. No. 12/114,490, entitled Skin Impedance Matching System and Method for Skin/Electrode Interface, the entire contents of which are hereby incorporated by reference in to this patent application.

The presence or absence of contractions on TOCO at the time of EMG measurement, as well as trans-vaginal cervical length and Bishop score (assessed no more than 24 hours before the EMG measurement), were also documented. As an example, student's t test and Mann Whitney U-test (when appropriate due to non-normal distribution of variables) were used on the EMG parameters to compare delivery within, vs. outside of, 24 hours from the measurement in term patients, and 7 days from the measurement in preterm patients. A p value of <0.05 was considered significant. Receiver operating characteristic (ROC) curves were used to estimate the predictive values of EMG parameters that were significantly higher in preterm patients delivering within 7 days. ROC analysis was also used to similarly assess the diagnostic accuracy of Bishop score, contractions on TOCO, and trans-vaginal cervical length for predicting preterm delivery within 7 days. Data on skin-electrode impedance and patients' BMI were analyzed by t-test and ANOVA to determine whether there were statistically significant (p<0.05) differences between groups with false positive, false negative, true positive, and true negative results. The Pearson correlation test was used to determine whether there was a correlation between patient's BMI and skin-electrode impedance overall. A p value of <0.05 was considered significant.

The software used for statistical analysis were SPSS 16.0 (SPSS Inc., Chicago, Ill., USA), True Epistat (Epistat Services, Richardson, Tex., USA), and SigmaStat 3.1 and SigmaPlot 9.0 (both from Systat software Gmbh, Erkrath, Germany).

To determine whether PV of electrical signals in the myometrium can be assessed non-invasively by uterine EMG, and whether this could be a useful parameter for characterizing labor, the study first compared PV of the EMG signals in term patients in labor (delivering within 24 hours from the EMG measurement, n=22) and non-labor (presenting with contractions but eventually delivering outside of 24 hours from the EMG measurement, n=6). Gestational age at inclusion did not differ significantly between the two groups (p=0.216). The median gestational age for labor patients was 39 2/7 (range 38 0/7 to 40 6/7 weeks) and for non-labor patients 38 5/7 (range 37 1/7 to 41 1/7 weeks). The median measurement to delivery interval for non-labor patients was 8 days (range 3 to 14 days) and in labor group 4 hours (range 2 to 14 hours).

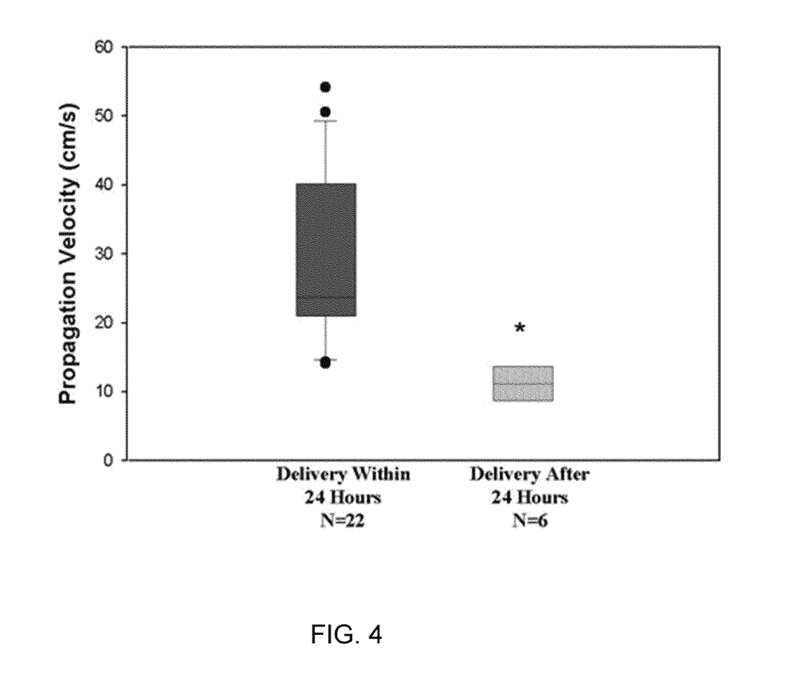

FIG. 4 shows that the measured PV was significantly higher (P<0.001) in labor (31.25±14.91 cm/s) compared with non-labor patients (11.31±2.89 cm/s). In an ROC analysis to distinguish between patients in true labor at term and false labor, PV had an area under the curve (AUC) of 0.98. For predicting delivery within 24 hours a PV>13.19 cm/s had 100% sensitivity, 83% specificity, 96% positive predictive value (PPV) and 100% negative predictive value (NPV). FIG. 2 illustrates a comparison of EMG propagation velocity values for term patients delivering within 24 hours of measurement with those delivering more than 24 hours from measurement. Propagation velocity was significantly higher (P<0.001) in the 24-or-fewer-hours group. Data are presented as error bars (median value, 10^{th}, 25^{th}, 75^{th} and 90^{th} percentile are plotted); *represents statistical significance (p<0.05).

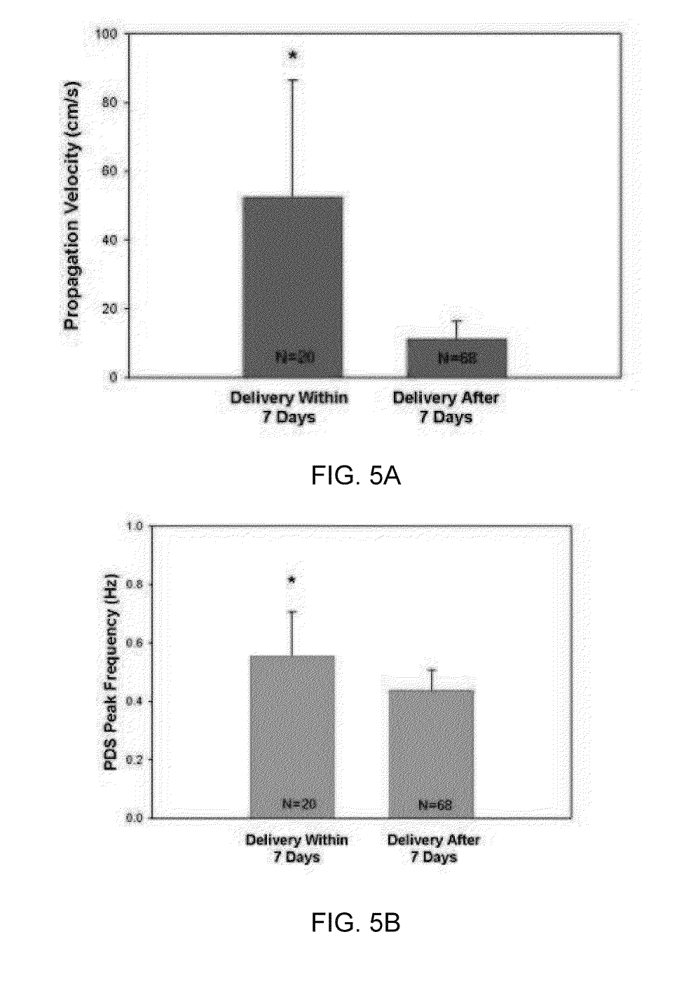

The results for the testing were as follows for preterm patients: The study subsequently determined whether PV may also be evaluated in patients presenting with signs and symptoms of preterm labor and its potential predictive value for preterm delivery. The study population consisted of 88 pregnant women admitted at our institution with the diagnosis of preterm labor at less than 34 weeks gestation. Patients were included in the study at a median of 28 5/7 weeks of gestational age (range 21 5/7 to 33 6/7 weeks). Delivery within 7 days from the EMG measurement occurred in 23% (20/88) of the cases. Clinical background variables are summarized in Table 1 as presented in FIG. 5.

Women who delivered within 7 days from the measurement did not differ from those who delivered later in regard to age, number of fetuses, parity, number of previous gestations, number of previous preterm deliveries, preterm premature rupture of membranes, smoking habits, illicit drugs abuse, tocolytic treatment, antenatal corticosteroid application or gestational age at study inclusion. Bishop score was significantly higher in women who delivered within 7 days (median score 7) compared with women who did not (median score 5) (p=0.01).

The groups did not differ regarding the presence of contractions on TOCO. Trans-vaginal cervical length was measured in 67% (59/88) of patients. Cervical length was not significantly shorter in women who delivered within 7 days (median 2.0 cm) compared with that in women who did not (median 2.8 cm) (p=0.16). Fetal fibronectin test was only performed in 26 (30%) patients. 62 (70%) of patients had at least one of the conditions that typically compromise the accuracy of the test, i.e., a digital cervical exam, collection of culture specimens, or vaginal probe ultrasound exam prior to referral to our institution, sexual intercourse within 24 hours prior to admission, rupture of membranes or advanced cervical dilation (3 cm or greater). It was positive in 10 women, of which only 2 delivered within 7 days. However, no woman with a negative test delivered within 7 days. The fibronectin test was done in only 2 of 20 patients who eventually delivered within 7 days, therefore a more rigorous statistical comparison of true preterm labor and false labor groups for fetal fibronectin was not possible.

TABLE 1

| | Women Delivering | Women Delivering | |
|---|---|---|---|
| Variable | Within 7 days (n = 20) | After 7 days (n = 68) | p |
| Maternal age | 24 (18-40) | 27 (18-43) | 0.59 |
| (years) | | | |
| Nulliparous | 5 | 16 | 0.99 |
| (n = 19) | | | |
| Number of | 1 (0-8) | 1 (0-11) | 0.64 |
| previous | | | |
| gestations | | | |
| Previous preterm | 2 | 13 | 0.54 |
| delivery or late | | | |
| abortion | | | |
| Twin gestations | 1 | 8 | 0.65 |
| Gestational age | 27 5/7 | 28 6/7 | 0.51 |
| at measurement | (22 6/7 to 33 4/7) | (21 5/7 to 33 6/7) | |
| Preterm | 3 | 2 | 0.42 |
| premature | | | |
| rupture of | | | |
| membranes | | | |
| Smoking | 1 | 9 | 0.58 |
| Illicit drug abuse | 1 | 7 | 0.72 |
| Tocolytic | 16 | 53 | 0.89 |
| treatment | | | |
| Antenatal | 11 | 54 | 0.09 |
| corticosteroids | | | |
| Contractions on | 7 | 19 | 0.64 |
| TOCO | | | |
| Bishop score | 7 (2-13) | 5 (1-10) | 0.01* |
| Transvaginal | 2.0 (0.5-3.5) | 2.8 (0.3-4.8) | 0.16 |
| cervical length | (n = 7) | (n = 52) | |
| (cm) (n = 59) | | | |

Data are median (range) and n. P value calculated by Mann-Whitney U-test and Student's T-test.
*represents statistical significance (p < 0.05).

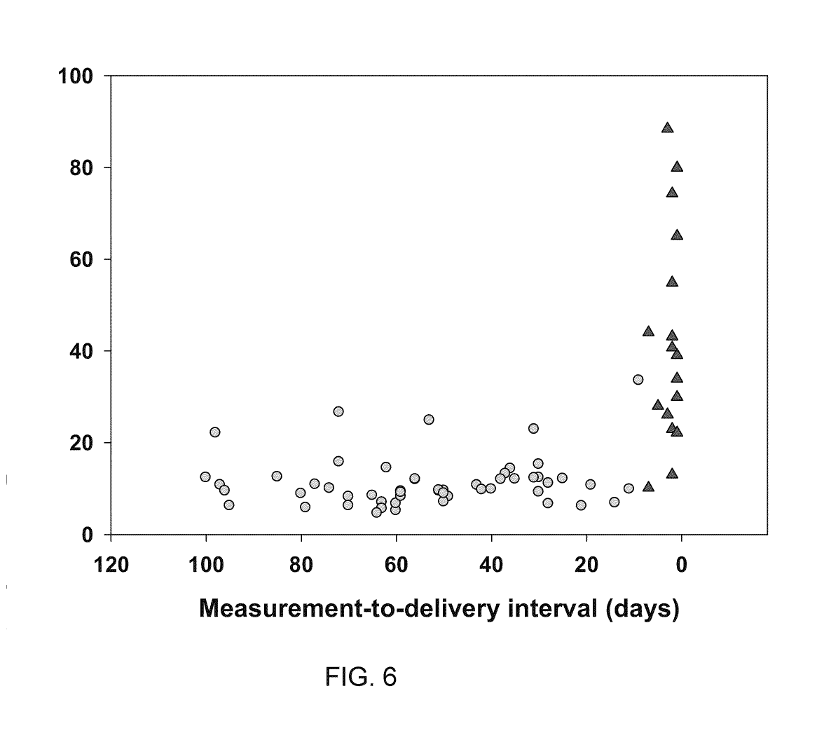

Table 1 illustrates the clinical background variables in women delivering preterm within, as compared to after, 7 days from the EMG measurement. Further, EMG Parameters—EMG PV was significantly higher in patients delivering within 7 days from the measurement (52.56±33.94 cm/s) compared to those who delivered after 7 days (11.11±5.13 cm/s) (p<0.001; FIG. 5A). As shown in FIG. 6, PV increased as the measurement-to-delivery interval decreased. PS peak frequencies were also significantly higher in women who delivered within 7 days (0.56±0.15 Hz) compared to those who did not (0.44±0.07 Hz) (p=0.002; FIG. 5B). All other EMG parameters analyzed did not differ significantly among groups: PS median frequency: 0.64±0.12 Hz vs. 0.68±0.05 Hz, p=0.11; PS median amplitude: 16.27±44.14 μV² vs. 10.16±16.29 μV², p=0.58; PS peak amplitude: 50.98±84.70 μV² vs. 70.56±134.64 μV², p=0.63, burst duration: 35.53±9.00 s vs. 39.32±12.26 s, p=0.21; inter-burst interval duration: 307.5±178.38 s vs. 348.96±227.27 s, p=0.65; standard deviation of burst duration: 7.44±5.85 s vs. 10.16±7.0 s, p=0.07; and standard deviation of inter-burst interval duration: 184.14±136.68 s vs. 149.76±157.92 s, p=0.26.

FIG. 5A illustrates a comparison of EMG propagation velocity values for preterm patients delivering within 7 days of measurement with those delivering more than 7 days from measurement, and FIG. 5B illustrates a comparison of EMG power spectrum (PS) peak frequency values for preterm patients delivering within 7 days of measurement with those delivering more than 7 days from measurement.

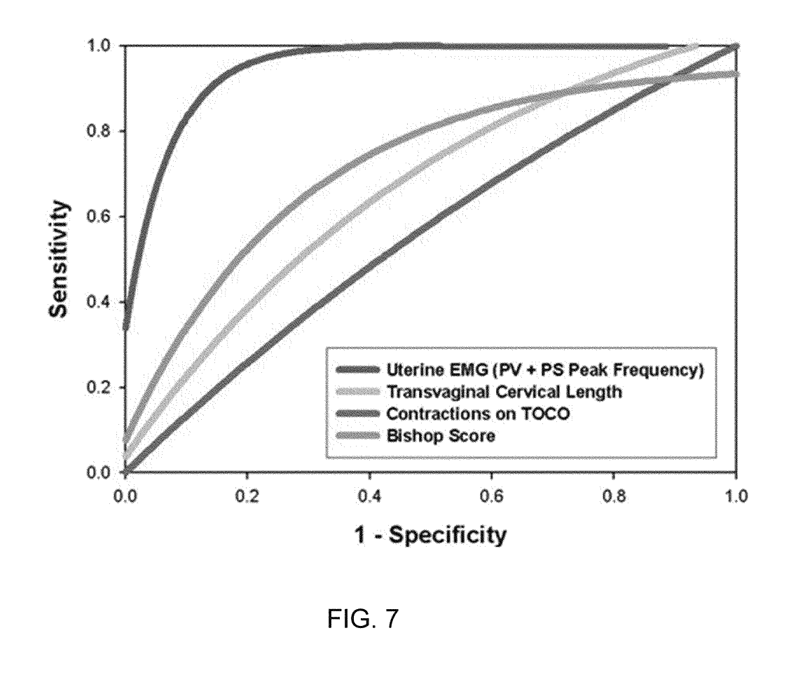

FIG. 7 illustrates EMG propagation velocity increased as the measurement-to-delivery interval decreased. Predictive values of EMG PV, PS peak frequency, and the combination (resealed sum) of these parameters for predicting preterm delivery at various time points were calculated (Table 2). ROC curves were generated for 1 day, 2 days, 4 days, 7 days, and 14 days to delivery. At 4 days to delivery, area under the curve (AUC) value was highest for PV, whereas for 7 days to delivery, PS peak frequency AUC value was highest. PV and PS peak frequency were then combined, by looking at the sum of their rescaled values. Specifically, PS peak frequency was multiplied by 100 and added to the corresponding PV value. The combination of these two parameters yielded the best predictive values at 7 days to delivery than for any parameter alone at any time point. A similar combination (product) using PV and PS peak frequency yielded no better results.

TABLE 2

| | 1 day to delivery | | | 2 days to delivery | | | 4 days to delivery | | | 7 days to delivery | | | 14 days to delivery | | |
|---|---|---|---|---|---|---|---|---|---|---|---|---|---|---|---|
| | | | PV + | | | PV + | | | PV + | | | PV + | | | PV + |
| | PV | PFr | PFr | PV | PFr | PFr | PV | PFr | PFr | PV | PFr | PFr | PV | PFr | PFr |
| AUC | 0.91 | 0.61 | 0.90 | 0.92 | 0.66 | 0.90 | 0.96 | 0.74 | 0.95 | 0.95 | 0.78 | 0.96 | 0.89 | 0.71 | 0.89 |
| Best cut- | 22.13 | 0.87 | 191.96 | 28.00 | 0.87 | 191.96 | 24.88 | 0.64 | 95.33 | 22.88 | 0.64 | 84.48 | 26.6 | 0.64 | 84.48 |
| off | cm/s | Hz | | cm/s | Hz | | cm/s | Hz | | cm/s | Hz | | cm/s | Hz | |
| Sensitivity | 100 | 14 | 14 | 0.77 | 8 | 8 | 82 | 18 | 53 | 85 | 15 | 70 | 70 | 13 | 61 |
| (%) | | | | | | | | | | | | | | | |
| Specificity | 80 | 100 | 100 | 0.92 | 100 | 100 | 93 | 100 | 100 | 94 | 100 | 100 | 99 | 100 | 100 |
| (%) | | | | | | | | | | | | | | | |
| PPV (%) | 30 | 100 | 100 | 0.63 | 100 | 100 | 74 | 100 | 100 | 81 | 100 | 100 | 94 | 100 | 100 |
| NPV (%) | 100 | 92 | 92 | 0.96 | 83 | 84 | 69 | 80 | 88 | 96 | 78 | 90 | 90 | 71 | 85 |

PV—propagation velocity;
PFr - PS peak frequency;
AUC—area under the curve;
PPV—positive predictive value;
NPV—negative predictive value;
Best cut-off values are presented as cm/s, Hz, and cm/s for PV, PFr, and their rescaled sum, respectively.

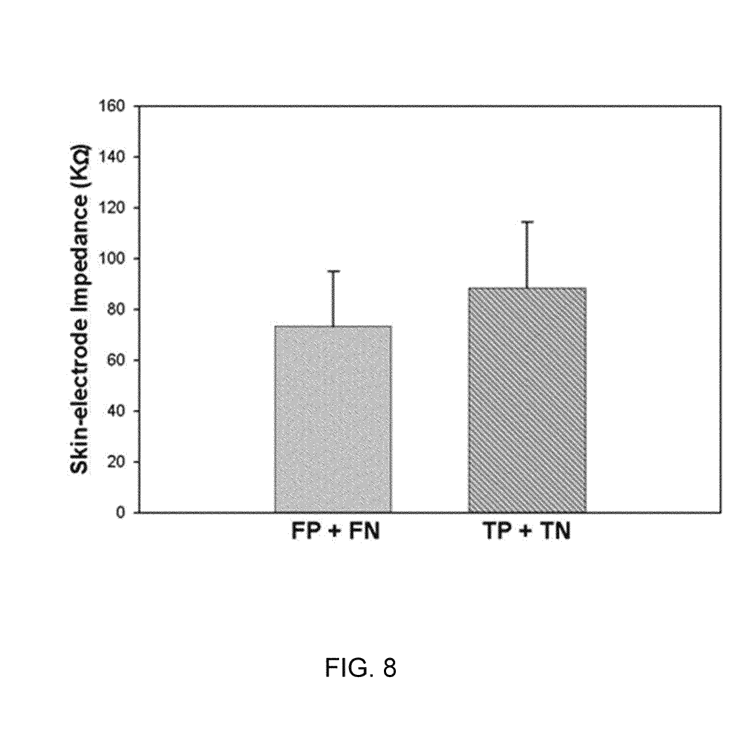

Table 2 illustrates predictive measures of EMG propagation velocity, PS peak frequency and the rescaled sum of these two parameters at 1, 2, 4, 7, and 14 days to delivery. FIG. 8 presents ROC curves illustrating predictive values of uterine EMG, i.e. Combination (rescaled sum) of PV and PS peak frequency, and three of the methods commonly used clinically to diagnose preterm labor: digital cervical examination (Bishop Score), transvaginal cervical length and presence of contractions on TOCO. Area under the curve (AUC), best cut-off value, sensitivity, specificity, negative predictive value (NPV) and positive predictive value (PPV) for EMG parameters and clinically used methods are shown in Table 3.

FIG. 8 illustrates a comparison of ROC curves for EMG parameters (combination of propagation velocity (PV) and PS peak frequency) and currently used methods to predict preterm delivery within 7 days.

TABLE 4

| Method | AUC | Best cut-off | Sensitivity | Specificity | PPV | NPV |
|---|---|---|---|---|---|---|
| EMG (PV + | 0.96 | 84.48 | 70% | 100% | 100% | 90% |
| PS Peak | | | | | | |
| Frequency) | | | | | | |
| Bishop Score | 0.72 | 10 | 18% | 100% | 100% | 81% |
| Transvaginal | 0.67 | 0.7 cm | 14% | 98% | 50% | 90% |
| Cervical | | | | | | |
| Length | | | | | | |
| Contractions | 0.54 | N/A | 35% | 72% | 27% | 79% |
| on TOCO | | | | | | |

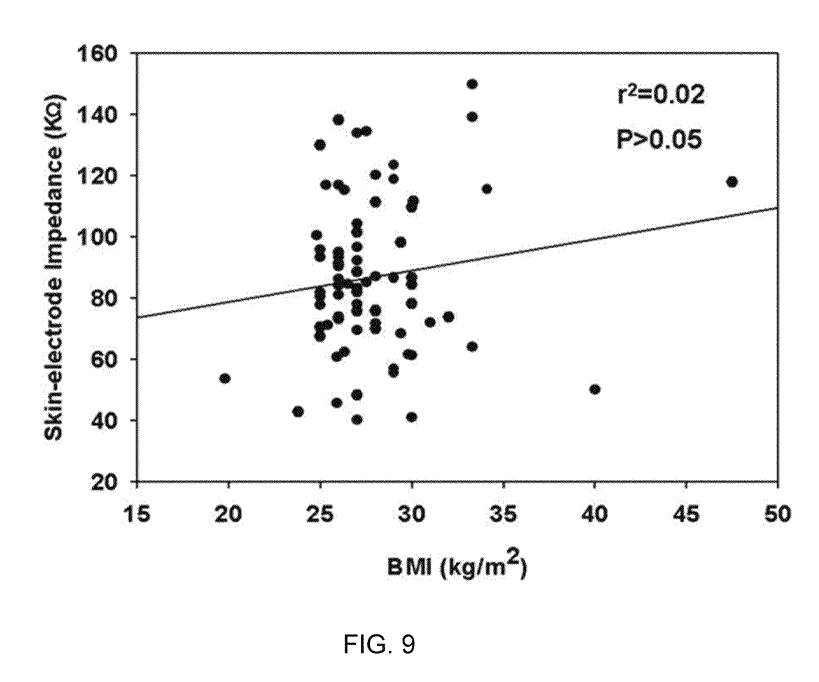

Table 4 illustrates predictive measures of EMG (combination of propagation velocity (PV) and PS peak frequency) parameters compared to currently used methods to predict preterm delivery within 7 days. The skin-electrode impedance is also a key to various embodiments of the disclosure. For example, 6 patients in preterm labor group (delivering within 7 days from the measurement) had a combination of PV and PS peak frequency lower than the best cut-off determined by the ROC analysis (false negative group). There were no false positive results. No significant differences in skin-electrode impedance measured before EMG recording, difference in impedance before and after recording or patients BMI was noted between these patients and preterm labor patients with high PV+PS peak frequency (true positive group) and/or non-labor patients with low PV+PS frequency (true negative group). There was also no significant correlation between skin-electrode impedance and patient's BMI overall (FIG. 9).

FIG. 9 illustrates a comparison of skin-electrode impedance measured before EMG recording between false positive (FP, N=0)+false negative (FN, N=6) and true positive (TP, N=14)+true negative (TN, N=68) groups (as determined by measurements of propagation velocity and PS peak frequency). There is no significant difference.

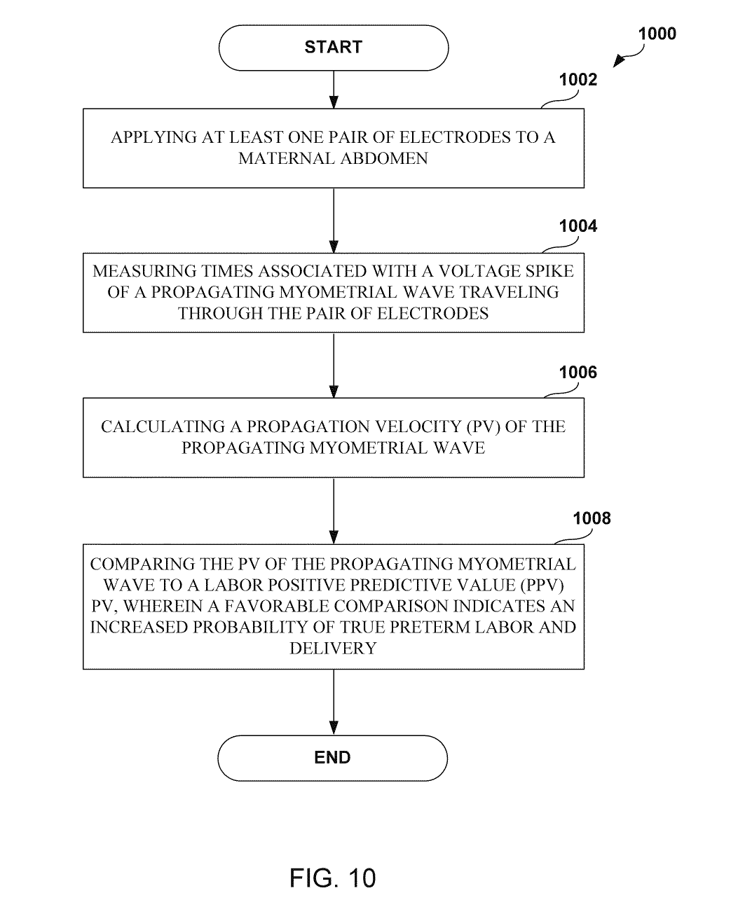

FIG. 10 illustrates that there is no significant correlation between skin-electrode impedance and patient's BMI. In view of the aforementioned experimental data, the study have concluded that regardless of the etiology of preterm labor, uterine contractions are associated with the common final pathogenetic pathway of prematurity. Techniques and methods for objectively monitoring uterine activity should, therefore, be useful, at least for identifying true preterm labor, if not also as screening tests for preterm birth. Currently, the most commonly used method to evaluate contractions is the TOCO. Unfortunately, this technique became a standard of care without ever undergoing vigorous clinical trials, in an age 40 years ago when such trials were in their infancy. TOCO measures the change in shape of the abdominal wall as a function of uterine contractions and, as a result, is a qualitative rather than quantitative method. It has been shown in several studies that monitoring uterine activity with TOCO is not helpful in identifying patients in preterm labor. Our present results also support this fact. Only 23% of patients with contractions on TOCO during the 30 minutes of EMG recording delivered within 7 days, and the absence of contractions apparently does not rule out preterm labor reliably, as the NPV is only 79%. Approximately 1 in 5 patients without contractions registering on TOCO did, nevertheless, deliver preterm within one week. It is unfortunate that clinicians still feel compelled to cling to this crude technology for assessing contractile activity, mainly because it is what is familiar, and because it is what is taught in medical school.

Embodiments of the present disclosure provide a method of measuring uterine electrical activity for the detection of uterine contractions that is superior to TOCO. First of all, it has been demonstrated by several studies that measuring uterine EMG activity has similar effectiveness of simple detection of uterine contractions as does TOCO, and even as compared to intrauterine pressure catheter, or IUPC. Secondly, different uterine EMG parameters can indicate myometrial properties that distinguish physiological preterm contractions from true preterm labor, which is something that the other devices cannot do. Finally, there are other advantages of uterine EMG over TOCO: EMG electrodes are generally considered by patients to be much more comfortable than TOCO belts, EMG electrodes do not require frequent repositioning when a patient is moving, and they are disposable, so that they do not contribute to cross contamination.

Of all of the possible EMG diagnostic variables, “timing related” EMG parameters seem to have the least predictive value. The study analyzed duration of uterine EMG bursts, inter-burst interval duration (which is inversely proportional to the frequency of the bursts) and the standard deviation of burst and inter-burst interval duration. None of these parameters differ significantly between the group of preterm patients who delivered within 7 days and those who did not. This is not in accordance with some studies, which found that the standard deviation of burst duration was smaller, and the frequency of burst was higher in labor patients. The study did, however, confirm the findings of Leman et al. and Buhimschi et al., who observed no differences in burst duration between preterm labor patients and women with preterm contractions that did not deliver preterm. Burst duration and frequency of bursts are the electrical equivalent of the duration and frequency of contractions, and these, not coincidentally, are the only properties of contractions that can be evaluated by TOCO. Thus, their poor predictive values are not surprising. Another type of EMG parameter can be categorized as “amplitude related”. Such parameters may represent the uterine EMG signal power, or alternatively, the EMG signal energy. Buhimschi demonstrated that an increase in PS peak amplitude precedes delivery (40). Other studies did not confirm these findings. In the present study, neither PS peak amplitude nor PS median amplitude is significantly higher in patients who delivered within 7 days compared to those who did not. It has been suggested that the major limitation of “amplitude related” EMG parameters is the fact that attenuation of myometrial signals occurs more for some patients and less for others, depending on a variance in subcutaneous tissues, and a variance in conductivity at the skin-electrode interface. Although in this study there are no significant differences in the preterm labor vs. Preterm non-labor groups in regard to patient's BMI and skin-electrode impedance, there could still be a difference in the individual pathways that multiple EMG signals traveled from the myometrium to the electrodes. This is especially plausible when one considers the relatively small size of the uterus at the gestational ages less than 34 weeks. In our opinion, these limitations make the “amplitude related” EMG parameters interesting but perhaps less reliable in the prediction of preterm labor.

The third group of EMG parameters can be defined as “frequency related” parameters. In the present study, the study focuses on PS median and peak frequency. Median frequency, although usually the most important parameter in the analysis of the striated muscle EMG, is rarely reported to be useful in the uterine EMG literature. The reason for that is probably the difference in the PS of the signals from the uterine and striated muscle cells. The PS of a striated muscle covers a broad frequency range (20 Hz-400 Hz), with a more or less bell-shaped distribution of signal energy. Thus, for striated muscle, the median frequency is a most useful parameter in the analysis of these signals. On the other hand, uterine EMG signals are filtered in order to exclude most components of motion, respiration, and cardiac signals, which yield a narrow “uterine-specific” band of 0.34 to 1.00 Hz. In this narrow frequency band produced by the uterus, the location of the power peak differs from one recording to another, and there are often competing “lesser” power-spectral peaks, not generally of consequence in the broad power-spectra of striated muscle.

This suggests that the type of narrow-band power distribution found in the uterine-specific range of frequencies may render using the median frequency a less useful parameter for characterizing the uterine electrical signals. Verdenik have, however, reported that as pregnancy approaches term, the median frequency of the uterine electrical activity becomes lower. It is not clear why this should be so, since other literature supports shifts to higher frequencies as a transition to labor occurs (59). Furthermore, shifts to lower median frequency are generally attributed to fatigue (57). A possible explanation for this is that the median PS frequency for the whole 30 minutes EMG recording and not for each burst separately was analyzed in that study. It may be that including non-uterine related electrical information (from the large portions of the recordings “in-between” bursts) contributed somehow to this result. In our study, wherein the study analyzed only the uterine-related electrical burst activity, the median frequency is not significantly different between the preterm labor and non-labor group.

Of the various EMG parameters previously used, PS peak frequency has been the most predictive of true labor in both human and animal studies. Shifts to higher uterine electrical signal frequencies occur during transition from a non-labor state to both term and preterm labor states, and can be reliably assessed by non-invasive trans-abdominal uterine EMG measurement. This is in accordance with the present study. PS peak frequency is significantly higher in the group of women who delivered within 7 days from the EMG measurement. It has also been shown previously by our group that PS peak frequency increases as the measurement-to-delivery interval decreases. The best predictive values of PS peak frequency have been identified at different measurement-to-delivery intervals by different authors (32, 33). The study finds the best values predicting delivery within 7 days as compared to those who did not. Embodiments of the present disclosure also demonstrate that PS peak frequency alone identifies patients in true preterm labor better than any other method currently available clinically.

Embodiments of the present disclosure introduce a new EMG parameter: the PV of uterine EMG signals. It has been shown in-vitro that the PV of electrical events in the myometrium is increased at delivery when gap junctions are increased. As a result of these findings, it has been suggested several times that EMG could be used to assess the PV in vivo, but the method to do this has not been described yet, and neither has the prognostic capability of PV for predicting labor (term or preterm) been evaluated.

Embodiments of the present disclosure not only demonstrate that PV of the electrical signals can be assessed from the non-invasive uterine EMG recording, but the Embodiments of the present disclosure may also use PV to predict preterm delivery more accurately than any other EMG parameter described so far, and certainly much more reliably than the methods used in everyday clinical practice. Because the embodiments of the present disclosure utilize an electrode and amplifier setup that increases the signal uterine electrical signal quality, this consequently resulted in an underestimation of the electrical signal time of arrival interval between electrodes. This, in turn, necessarily produces a propagation velocity overestimation. This overestimation occurs for both labor and non-labor patients alike, since it is a systematic error, and so significant differences in the electrical signal time of arrival interval between true labor and false labor patients both at term and preterm using this arrangement are seen. More importantly, because the propagation velocity is proportional to this time interval, the velocity estimation, mathematically speaking, is also significantly different. Future studies may utilize different electrode and amplifier configurations to more accurately pin down the uterine electrical signal propagation velocity value and directionality.

By “combining” the PV and PS peak frequency, the embodiments of the present disclosure provide a model that more accurately predicts spontaneous preterm birth. The ROC-curve analysis for this model has an AUC of 0.96. This makes this methodology extremely valuable in everyday clinical practice. When uterine EMG is measured in patients presenting with signs and symptoms of preterm labor and the combination (sum) of PV and PS peak frequency exceeds the cut-off value of 84.48 this predicts delivery within 7 days with a 100% certainty according to study data (PPV=100% in 88 patients). EMG does, therefore, identify the patients in true preterm labor very reliably. These patients and their babies are the ones who really benefit from early institution of tocolytic therapy, transport to a hospital with facilities for neonatal intensive care, administration of steroids, and antibiotics. At the same time, this methodology also identifies patients in false preterm labor who are not going to deliver within the next 7 days. It can, therefore, help to avoid substantial economic costs associated with hospitalization, the maternal risks associated with tocolytics, and the potential fetal risks associated with steroids. In the case of low PV+PS peak frequency values, it therefore stands to reason that it would be safe not to admit, treat, or transfer the patient, regardless of the presence of contractions on TOCO, and regardless of digital cervical exam and transvaginal cervical length results, since the changes in the myometrium required for labor are not yet even established.

It is also important to point out that the study focused on preterm delivery before 34 weeks', when the incidence of fetal death and handicap is mainly increased (45). Attempts to stop preterm labor are rarely made after this gestational age, and distinguishing true preterm labor from physiologic contractions is therefore of clinical importance especially at these earlier gestations.

One of the potential limitations of the transabdominal uterine EMG could be its low sensitivity in recording contractions in patients with high BMI, as is the case with TOCO (53). Our studies, and those of others, have shown, however, that uterine EMG signals are minimally affected by the amount of subcutaneous fat tissue and transabdominal uterine EMG can monitor contractions in obese women better than the TOCO (38,53). The present study confirms this. Both PV and PS frequency are significantly higher in preterm labor patients, although patient's BMI is not significantly different in the labor and non-labor groups. Moreover, BMIs of patients included were as high as 47.5 kg/m ² (median 27 kg/m², range 19.5-47.5 kg/m²). Patient's BMI is also not correlated with skin electrode impedance measured before EMG recording and the fall in impedance during the recording.

This is in accordance with previously published studies, which suggested that the impedance is more a result of the type (material, size, and geometry) of electrodes used, skin temperature at the electrode and the galvanic skin response than the amount of adipose subcutaneous tissue. However, the study find the false negative results that the study observe (i.e., low PV and/or PS peak frequency values in patients in true labor) also cannot be attributed to high skin-electrode impedance. This suggests that the false negative results do not represent the failure of the transabdominal EMG instrument to detect uterine electrical activity reliably, but rather are either a consequence of myometrial physiology or are of an inherent limitation of the signal processing technique.

It has been reported previously by our group, that the uterus shows high-frequency activity only about 10% to 20% of the time when far removed from delivery and it shows high-frequency activity about 80% to 90% of the time when within 24 hours of delivery for term patients. The false negative results of PS peak frequency analysis can therefore be attributed to the possibility that even when the woman is measured close to delivery she could be in a temporary “low-electrical-frequency state”. More work has to be done, however, to determine whether similar fluctuations of PV also occur.

All of the signal processing techniques used in our study are linear techniques. The uterus is, however, a complex non-linear dynamic system and non-linear signal processing techniques could potentially be very useful in analyzing such a system. Consequently the false negative results in our study can also be the result of the inability of the methods used to analyze the non-linear components of the uterine EMG signals. Studies have been done on some non-linear analysis techniques such as fractal dimension of the burst of electrical activity and calculation of the sample entropy of the signal yielding promising results. Although combination of PV and PS peak frequency differentiates preterm patients in true labor from those in false labor more reliably than any method available today, the addition of non-linear parameters could make this model even more effective.

Another potential limitation of this study is the use of tocolytics, which can affect uterine activity by several different mechanisms, and can possibly inhibit uterine EMG activity by themselves. However, there is no significant difference in the use of tocolytics in the group of women who delivered within 7 days as compared to those who did not. It is, consequently, very unlikely that the use of tocolytics is a significant confounding factor in our study, but more work should be done to answer this and related questions.

Similar to uterine contractions, the phenomenon of disruption of the extra-cellular matrix within the cervix occurs in every preterm labor, regardless of its etiology. However, the methods currently available to clinicians to assess these changes in the cervix have several major drawbacks. For example, digital cervical examination is subjective, and does not provide accurate diagnosis of true preterm labor. Our present findings are in accordance with this: the predictive measures of Bishop Score were high only at scores of >10, which is not useful clinically, because at that point imminent delivery is already obvious. In contrast with several studies, the predictive value of trans-vaginal cervical length is not better, in fact is even worse, than that of the Bishop score as shown by this study. It can be argued that the cervical length was only measured in two thirds of the patients included in our study and only in 7 patients who delivered within 7 days.

In many of the patients who presented with advanced cervical dilatation, cervical length was not obtained, and those patients were more likely to deliver within 7 days. The predictive values would, therefore, most likely be better if the transvaginal cervical length of all patients were known. However, several patients with short cervices in this study did not deliver within one week, and some did not deliver preterm at all. This illustrates what has been described before: the value of trans-vaginal cervical length lies in its high NPV, while it does not identify patients in true preterm labor reliably.

Previous studies documented evidence that cervical collagen content can be monitored non-invasively measuring light-induced fluorescence (LIF) of collagen. This method could detect the change in the composition of the cervix, regardless of its length. The combination of uterine EMG, which identifies myometrial preparedness to labor, and cervical LIF, which objectively assesses the change in cervical structure, has the potential to answer one of the biggest questions in obstetrics today: how to identify patients in true preterm labor who benefit from tocolytic and steroid therapy, and at the same time avoid side effects and costs of treatment in patients in false labor.

FIG. 10 provides a logic flow diagram of a method of predicting true preterm labor and delivery in accordance with embodiments of the present disclosure. Operations 1000 begin with applying at least one pair of electrodes to a maternal abdomen in block 1002. The time associated with measuring a voltage spike of a propagating myometrial wave traveling through the pairs of electrodes are recorded in block 1004. These times allow the amount of time required for the propagating myometrial wave to transverse the distance between electrodes to be determined. With this information a propagation velocity (PV) of the propagating myometrial wave may be determined in block 1006. This PV may be compared to a labor positive predictive value (PPV) in block 1008. A favorable comparison indicates an increased probability of true preterm labor and delivery. The propagating myometrial wave may be detected using electrodes to detect a uterine electromyography (EMG) signal associated with the propagating myometrial wave. This increased probability of true preterm labor may especially indicate and favorably predict delivery within seven days. In addition to the PV signal a power spectrum signal may be measured and used to determine the increased probability of true preterm labor and delivery. The power spectrum signal may be analyzed for peak and median frequency, peak and medium amplitude, restoration, inter burst interval duration, and standard deviation of inter burst interval duration.

The foregoing has outlined features of several embodiments so that those skilled in the art may better understand the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A method operable to predict True Preterm Labor and Delivery comprising:
applying at least two pairs of electrodes to a maternal abdomen;
measuring a first time associated with a voltage spike of a propagating myometrial wave traveling through a first electrode pair of the at least two pairs of electrodes;
measuring a second time associated with the voltage spike of the propagating myometrial wave traveling through a second electrode pair of the at least two pairs of electrodes
determining an amount of time (T) required for the propagating myometrial wave to traverse a distance (D) between the first and the second electrode pairs;
calculating a propagation velocity (PV) of the propagating myometrial wave; and
comparing the PV of the propagating myometrial wave to a labor positive predictive value (PPV) PV, wherein a favorable comparison between the PPV PV and the calculated PV indicates an increased probability of True Preterm Labor and Delivery.

2. The method of claim 1, wherein the at least two pairs of electrodes detect a uterine electromyography (EMG) signal associated with the propagating myometrial wave.

3. The method of claim 1, wherein the amount of time is determined by a time difference associated with the voltage spikes detection at the at least two pairs of electrodes attached to a maternal abdomen.

4. The method of claim 1, wherein the increased probability of True Preterm Labor and Delivery results in delivery within a predefined number of days.

5. The method of claim 1, further comprising measuring a power spectrum (PS) signal of the propagating myometrial wave.

6. The method of claim 5, wherein the PS signal is analyzed from about 0.34 to 1.00 Hz.

7. The method of claim 5, wherein the PS signal is analyzed for:
PS peak and median frequency;
PS peak and median amplitude;
burst duration;
interburst interval duration; and
standard deviation of burst and interburst interval duration.

8. The method of claim 1, wherein calculating the PV of the propagating myometrial wave comprises dividing the distance (D) that the propagating myometrial wave travels by the amount of time (T).

9. The method of claim 1, further comprising matching a skin impedance associated with the maternal abdomen.

10. The method of claim 1, wherein an unfavorable comparison between the PPV PV and the calculated PV indicates a decreased probability of True Preterm Labor and Delivery.

11. A system operable to predict True Preterm Labor and Delivery, the system comprising:
at least two pairs of electrodes in communication with a maternal abdomen, the pairs of electrodes operable to acquire raw uterine electromyography (EMG) signals associated with a propagating myometrial wave;
a signal processing module communicably coupled to the pairs of electrodes, the signal processing module operable to:
filter and amplify the raw uterine EMG signals;
obtain a processed EMG signal;
calculate a propagation velocity (PV) of the propagating myometrial wave based on the processed EMG signal and a known location of the pair of electrodes;
comparing the PV of the propagating myometrial wave to a labor positive predictive value (PPV) PV, wherein a favorable comparison between the PPV PV and the calculated PV indicates an increased probability of True Preterm Labor and Delivery; and
display a communication indicating the increased probability of True Preterm Labor and Delivery.

12. The system of claim 11, further comprising a skin impedance matching system, the skin impedance matching system operable to correct the processed EMG signal for a skin impedance associated with the a maternal abdomen.

13. The system of claim 12, the skin impedance matching system comprising:
a matching module configured to determine the skin impedance by sensing an input impedance from the patient through the pair of electrodes, and amplifying and digitizing the input impedance;
a resistor ladder network configured to match the skin impedance using at least one resistor;
a microprocessor configured to analyze the input impedance and generate a series of control signals to direct the resistor ladder network to match the skin impedance; and
a sensing module configured to sense uterine EMG signals from the patient through the pair of electrodes in conjunction with the resistor ladder network.

14. The system of claim 13 in which the resistor ladder network matching is accomplished through a software calibration factor.

15. The system of claim 14, wherein the sensing module is communicably coupled to the signal processing module.

16. The system of claim 11, wherein signal processing module is operable to determine an amount of time (T) required for the propagating myometrial wave to traverse a distance (D) between the pair of electrodes in communication with a maternal abdomen, the amount of time is determined by a time difference associated with the voltage spikes at the at least two pairs of electrodes.

17. The system of claim 11, wherein the increased probability of True Preterm Labor and Delivery results in delivery within a predefined number of days.

18. The system of claim 11, further comprising a sensing module operable to measure a power spectrum (PS) signal of the propagating myometrial wave.

19. The system of claim 18, wherein the PS signal is analyzed from about 0.34 to 1.00 Hz.

20. The system of claim 18, wherein the PS signal is analyzed for:
PS peak and median frequency;
PS peak and median amplitude;
burst duration;
interburst interval duration; and
standard deviation of burst and interburst interval duration.

21. The system of claim 11, wherein calculating the PV of the propagating myometrial wave comprises dividing the distance (D) that the propagating myometrial wave travels by the amount of time (T).

22. The system of claim 11, further comprising matching a skin impedance associated with the maternal abdomen.

23. The system of claim 11, wherein an unfavorable comparison between the PPV PV and the calculated PV indicates a decreased probability of True Preterm Labor and Delivery.
